(19) United States
(12) Patent Application Publication
(10) Pub. No.: US 2012/0029525 A1
(43) Pub. Date: Feb. 2, 2012

(54) MEDICAL DEVICE AND METHOD FOR VASCULAR ACCESS

(76) Inventor: J. Pedro FROMMER, Houston, TX (US)

(21) Appl. No.: 12/844,341

(22) Filed: Jul. 27, 2010

(51) Int. Cl. A61M 25/00

(52) U.S. Cl. 606/108; 604/524

(57) ABSTRACT

A medical device comprising a rod which includes an elongate rod body and a rod handle. The rod body is composed of a biocompatible semi-rigid material and is curved from between 75 to 105 degrees relative to the longitudinal axis of the rod body. The distal end of the rod body is beveled to a point so that it is capable of puncturing through human skin. The medical device also comprises a sheath which includes a hollow sheath body and a sheath handle. The sheath body is straight. The rod body is capable of being inserted into the sheath body and is able to slide along the sheath body's longitudinal axis.

### BACKGROUND

Hemodialysis is used to provide artificial replacement of lost kidney function caused by certain kidney diseases. The process consists in recirculating the patient's blood through a dialysis filter in order to remove toxins and excess fluid. In order to do this procedure, the patient needs to be connected to the hemodialysis machine via an access. This is done by either an access in the patient's limb (fistula or graft), or a temporary catheter inserted into the patient's central vein. The safest, most efficient and most common access is a permanent access in a limb. For this purpose, a surgeon will join an artery with a vein, either directly (fistula) or through a synthetic tube of PTFE (graft). The dialysis personnel then will insert two large bore needles connected to the tubing that takes the blood to the machine for the dialysis process. This procedure is performed for 3 to 4 hours, three times a week. It is a matter of time that, due to repeated needle punctures and other factors, the access will malfunction, or stop functioning altogether, and clot. The most common cause for this malfunction is a critical narrowing of the access to the point of slowing down or stopping the circulation of blood. At this point, the patient cannot be dialysed and access viability needs to be restored. The state of the art procedure to restore blood flow to a malfunctioning or clotted access is by inserting two sheaths at opposite ends of the access, and using embolectomy catheters, and angioplasty balloons, remove the clots, and dilate the narrowed blood vessels. This is done under fluoroscopy which exposes the operator and the assistants to radiation. The initial insertion of the first sheath is usually easy, since that segment of the access is better exposed. The second insertion can sometimes be very difficult since the area is sometimes less exposed, specifically in the upper arm, or thighs of patients. When this happens, the time of the procedure is prolonged significantly, and so is the exposure to radiation. The present medical device has the purpose of circumventing this problem by using the first insertion to exteriorize a needle at the desired site of the second insertion and using this guide to enter the access for the second time thus significantly shortening procedure time and radiation exposure in difficult cases.

### SUMMARY

The problems noted above are solved in large part by a medical device and method. In some embodiments, a medical device comprises a rod which includes an elongate rod body and a rod handle. The rod body is composed of a biocompatible semi-rigid material and is curved from between 75 to 105 degrees relative to the longitudinal axis of the rod body. The distal end of the rod body is beveled to a point so that it is capable of puncturing through human skin. The medical device also comprises a sheath which includes a hollow sheath body and a sheath handle. The sheath body is substantially straight. The rod body is capable of being inserted into the sheath body and is able to slide along the sheath body's longitudinal axis. In some embodiments the rod body may be composed of a Nitinol alloy and may be hollow. The sheath handle and rod handle may be composed of non-slip materials. Additionally, in some embodiments, the distal end of the rod body is superelastic.

Another illustrative embodiment includes a medical device comprising a rod which includes an elongate rod body and rod handle. The rod also includes a first position and a second position. The medical device also comprises a sheath which includes a sheath body and a sheath handle in which the sheath body is substantially straight. When the rod is in the first position, the distal end of the rod body is fully disposed within the sheath body. When the rod is in the second position, the distal end of the rod body is extended from the sheath body and oriented about 90 degrees relative to the longitudinal axis of the sheath body. In some embodiments, when the rod is in the first position, the rod handle is horizontally separated from the sheath handle. When the rod is in the second position, the rod handle is in contact with the sheath handle.

Yet another illustrative embodiment includes a method comprising making a first insertion on a patient. The method also comprises inserting a rod body entirely disposed within a sheath body into the patient through the first insertion. The method continues with sliding the rod body longitudinally along the sheath body until a distal end of the rod emerges from the sheath body at about a 90 degree angle relative to the longitudinal axis of the sheath body. The method also comprises puncturing the skin of the patient with the distal end of the rod body.

### BRIEF DESCRIPTION OF THE DRAWINGS

For a detailed description of various disclosed embodiments, reference will now be made to the accompanying drawings in which:

FIGS. 1a, 1b, and 1c show detailed views of a rod which may be used in assisting in making an insertion on a patient from inside the patent, in accordance with various embodiments of the invention;

FIGS. 2a, 2b, and 2c show detailed views of a sheath which may be used in assisting in making an insertion on a patient from inside the patent, in accordance with various embodiments of the invention;

FIG. 3 shows a detailed view of a rod and sheath in combination with the rod fully inside the sheath which may be used in assisting in making an insertion on a patient from inside the patent, in accordance with various embodiments of the invention;

FIG. 4 shows a detailed view of a rod and sheath in combination with the rod exiting the sheath which may be used in assisting in making an insertion on a patient from inside the patent, in accordance with various embodiments of the invention;

FIGS. 5a and 5b show a patient in which a rod and sheath combination is being used, in accordance with various embodiments of the invention;

FIG. 6 shows an illustrative flow diagram of a method implemented in accordance with embodiments of the invention.

### NOTATION AND NOMENCLATURE

Certain terms are used throughout the following description and claims to refer to particular device components. As one skilled in the art will appreciate, companies may refer to a component by different names. This document does not intend to distinguish between components that differ in name but not function. In the following discussion and in the claims, the terms “including” and “comprising” are used in an open-ended fashion, and thus should be interpreted to mean “including, but not limited to . . . .” Also, the term “couple” or “couples” is intended to mean either an indirect or direct connection. Thus, if a first device couples to a second device, that connection may be through a direct connection, or through an indirect connection via other devices and connections. Further, the terms “axial” and “axially” generally mean along or parallel to a central or longitudinal axis (e.g., the sheath body), while the terms “radial” and “radially” generally mean perpendicular to the central or longitudinal axis. For instance, an axial distance refers to a distance measured along or parallel to the central or longitudinal axis, and a radial distance refers to a distance measured perpendicularly from the central or longitudinal axis. Further, the terms “coaxial” and “coaxially” generally refer to the relative orientation of two structures or components that have coincident central or longitudinal axes.

### DETAILED DESCRIPTION

The following discussion is directed to various embodiments of the invention. Although one or more of these embodiments may be preferred, the embodiments disclosed should not be interpreted, or otherwise used, as limiting the scope of the disclosure, including the claims. In addition, one skilled in the art will understand that the following description has broad application, and the discussion of any embodiment is meant only to be exemplary of that embodiment, and not intended to suggest that the scope of the disclosure, including the claims, is limited to that embodiment.

Some embodiments include a device which is capable of being inserted into a patient's blood vessels, and, at a desired location, making a second insertion on the patient from within the patient himself/herself. This enables a physician to gain vascular access at a location in the body which may be difficult to obtain otherwise due to, for example, the size of the patient's body or appendage where the vascular access is required.

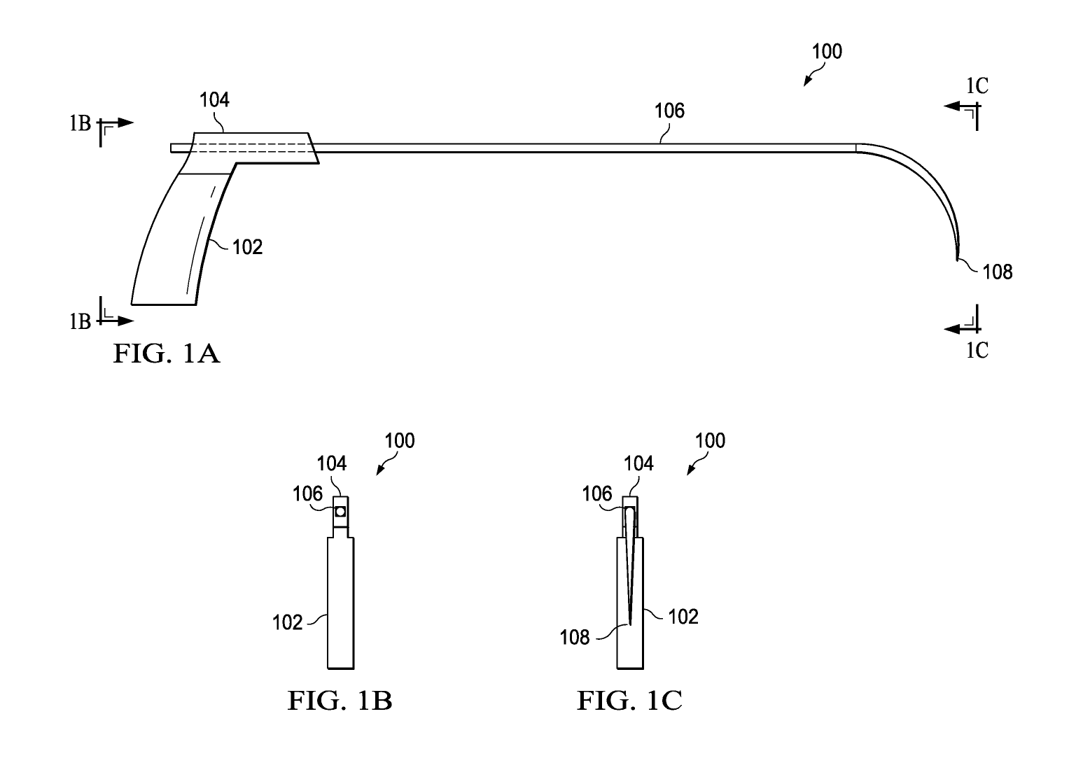

FIGS. 1a, 1b, and 1c show detailed views of a rod 100 which may be used in assisting in making an insertion on a patient from inside the patent, in accordance with various embodiments of the invention. FIG. 1a shows a detailed view of rod 100 as viewed as from the side, in accordance with various embodiments of the invention. Rod 100 is comprised of rod handle 102, rod housing 104, rod body 106, and distal end 108 which is a section of rod body 106. In some embodiments, rod handle 102 may be 5 centimeters in length and 2 centimeters in width; however, other lengths and widths may also be used in other embodiments, so long as a physician may hold and guide rod 100. Rod handle 102 preferably is manufactured of a non-slip material. That is, rod handle 102 is preferably manufactured with a material that will not slip in a physician's hand when rod handle 102 has body fluid (e.g. blood) on its surface. Some such materials include materials with a relatively high coefficient of friction, e.g. rubber, or a textured metal or plastic surface, e.g. ridged, knurled, or notched. However, rod handle 102 may have a smooth surface, so long as a physician may hold and guide rod 100.

Rod housing 104 is radially coupled to rod handle 102 and axially coupled to rod body 106. Rod housing 104 is tubular, such that rod body 106 may extend through it. Rod housing 104 may be 3 centimeters in length and 5 millimeters in width, although other lengths and widths may be used. Rod housing 104 preferable is square in shape, but may be of other shapes (e.g. circular, hexagonal, octagonal). Rod housing 104 may be made of any rigid material, such as stainless steel.

Rod body 106 is substantially straight until it reaches distal end 108 where it curves. In at least some embodiments, “substantially straight” means straight plus or minus three degrees. Rod body 106 preferably is smooth, so that it may slide easily along a sheath. Rod body 106 may be approximately 1 millimeter in diameter, and approximately 20 centimeters in length, although it may be more or less in both diameter and length. Rod body 106 may be hollow throughout its length, and thus tubular. Rod body 106 may be manufactured with any biocompatible material; so long as the material remains semi-rigid (e.g. a Nitinol alloy). Semi-rigid means that the material must be capable of bending under pressure, but remains rigid without pressure. Rod body 106 is axially coupled to rod housing 104 and radially coupled to rod handle 102 so that rod body 106 extends beyond rod housing 104 and rod handle 102.

Distal end 108 is a section of rod body 106 which is curved in the direction of rod handle 102. In some embodiments the length of distal end 108 may be 1.5 centimeters although the length may vary. Preferably, distal end 108 is radially curved approximately 90 degrees from the longitudinal axis of the rest of rod body 106. Thus, distal end 108 is preferably curved about 90 degrees relative to the longitudinal axis of the rest of rod body 106. However, distal end 108 may be radially curved as little as 75 degrees and as much as 105 degrees from the longitudinal axis of the rest of rod body 106. Like the other sections of rod body 106, distal end 108 is bendable, meaning that the angle of the curve may be increased or decreased. For example, distal end 108 may be bent such that the angle of the curve decreases to zero degrees relative to the longitudinal axis of the rest of rod body 106. However, distal end 108 will remain curved unless force is applied and will return to its original curved shape when the applied force is removed. This is sometimes referred to as superelasticity. Distal end 108 is beveled to a point, such that it is capable of puncturing through human skin and blood vessels.

FIGS. 1b and 1c show different cross sectional views of rod 100, in accordance with various embodiments of the invention. FIG. 1b is a cross sectional view from the rear of rod 100 longitudinal to rod body 106. As can be seen, rod body 106 extends through rod handle 102. Rod body 106 is a hollow tube such that a thin wire may extend through its shaft. FIG. 1c is a cross sectional view of rod 100 from its front longitudinal to rod body 106. Rod housing 104 is hollow so that rod body 106 extends through both sides of it. Distal end 108 comes to a point so that it may puncture through skin and blood vessels.

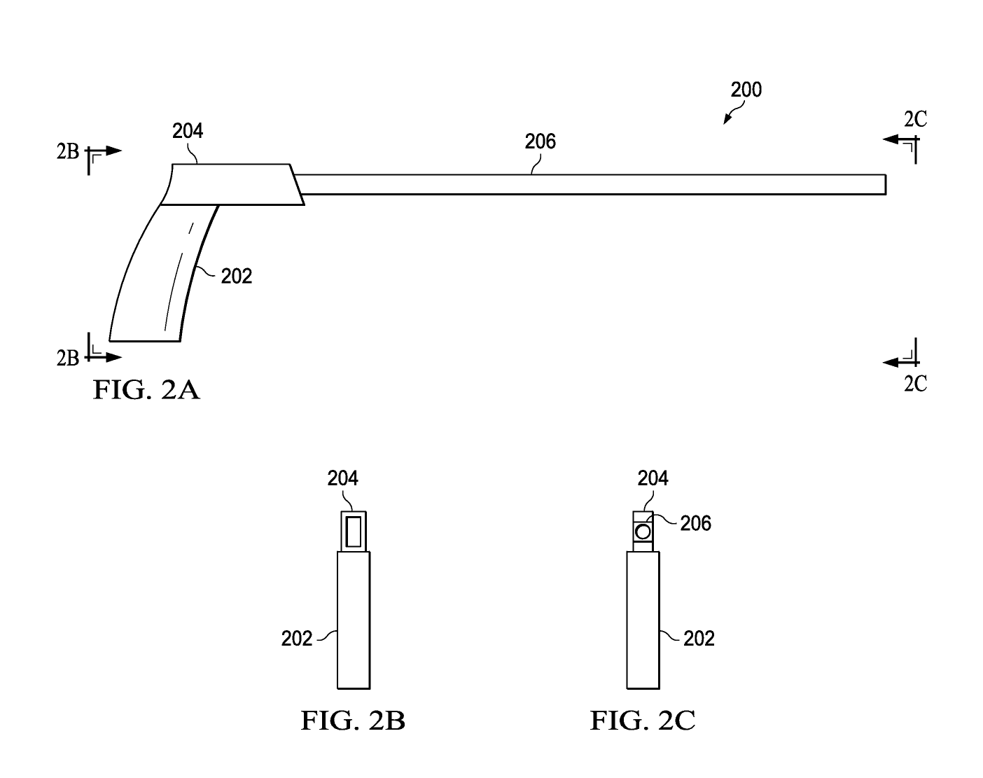

FIGS. 2a, 2b, and 2c show detailed views of a sheath 200 which may be used with rod 100 in assisting in making an insertion on a patient from inside the patent, in accordance with various embodiments of the invention. FIG. 2a shows a detailed view of sheath 200 as viewed from the side, in accordance with various embodiments of the invention. Sheath handle 202 may be 5 centimeters in length and 2 centimeters in width; however, other lengths and widths may also be used, so long as a physician may hold and guide sheath 200. Sheath handle 202 preferably is manufactured of a non-slip material. That is, sheath handle 202 is preferably manufactured with a material that will not slip in a physician's hand when sheath handle 202 has body fluid (e.g. blood) on its surface. Some such materials include materials with a relatively high coefficient of friction, e.g. rubber, or a textured metal or plastic surface, e.g. ridged, knurled, or notched. However, sheath handle 202 may have a smooth surface, so long as a physician may hold and guide sheath 200.

Sheath housing 204 is radially coupled to sheath handle 202 and axially coupled to sheath body 206. Sheath housing 204 is tubular, such that rod housing 104, from FIG. 1 may extend into it. However sheath housing 204 is closed on one end. Sheath housing 204 may be 3.5 centimeters in length and 5.5 millimeters in width, although other lengths and widths may be used. However, sheath housing 204 must be longer and wider than rod housing 104 so that rod housing 104 may be inserted into sheath housing 204. Sheath housing 204 preferable is square in shape; however it may be other shapes (e.g. circular, hexagonal, octagonal), so long as it is the same shape as rod housing 104. Sheath housing 204 may be made of any rigid material, such as stainless steel.

Sheath body 206 is substantially straight. Sheath body 206 preferable is circular in shape; however it may be other shapes (e.g. square, hexagonal, octagonal). Sheath body 206 may be 13 centimeters in length, but may also be shorter or longer. Sheath body 206 is typically shorter than rod body 106 from FIG. 1. Sheath body 206 may be made of an opaque plastic material or a metal, e.g. stainless steel, so that it is visible in x-rays and during fluoroscopy and so that it is rigid. Sheath body 206 is axially coupled to sheath housing 204 and radially coupled to sheath handle 202. Sheath body 206 is a hollow tube. Sheath body 206 may have a diameter of 2 millimeters, but other diameters may be used. However, sheath body 206 preferably has a diameter larger than rod body 106. from FIG. 1, such that rod body 106 may extend through sheath body 206.

FIGS. 2b and 2c show different cross sectional views of sheath 200, in accordance with various embodiments of the invention. FIG. 2b is a cross sectional view from the rear of sheath 200 longitudinal to sheath body 206. As can be seen, sheath housing 204 is coupled to sheath handle 202 and is a hollow tube such that rod housing 104, from FIG. 1 may be inserted. FIG. 2c is a cross sectional view of sheath 200 from its front longitudinal to sheath body 206. Sheath housing 204 is closed at the front end and coupled to sheath body 204.

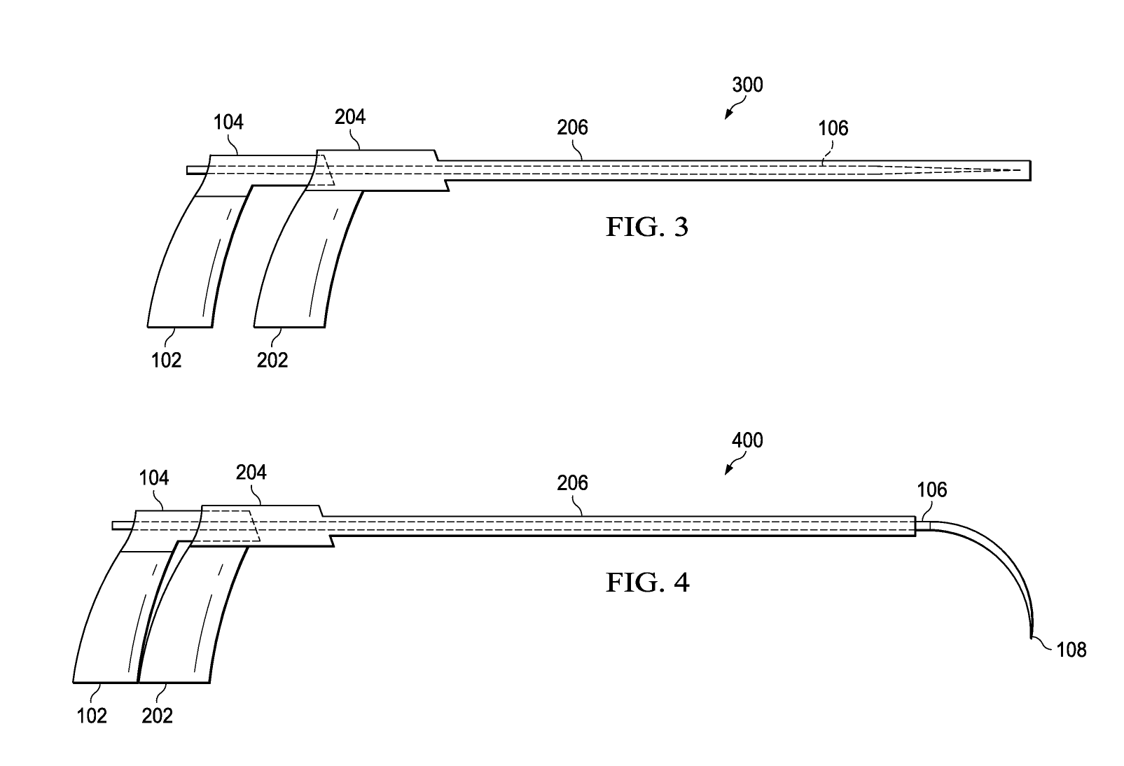

FIG. 3 shows a detailed view of rod 100 and sheath 200 in combination, such that rod body 106 is fully disposed within the sheath body 206, labeled vascular access device 300, which may be used in assisting in making an insertion on a patient from inside the patent, in accordance with various embodiments of the invention. Rod body 106 is inserted into sheath housing 204. Rod body 106 is then pushed the entire length of sheath body 206 until distal end 108 is near the end of sheath body 206. Distal end 108, while curved, is bendable, and thus capable of being inserted into the tubular sheath body 206. Rod housing 104 is then inserted into sheath housing 204.

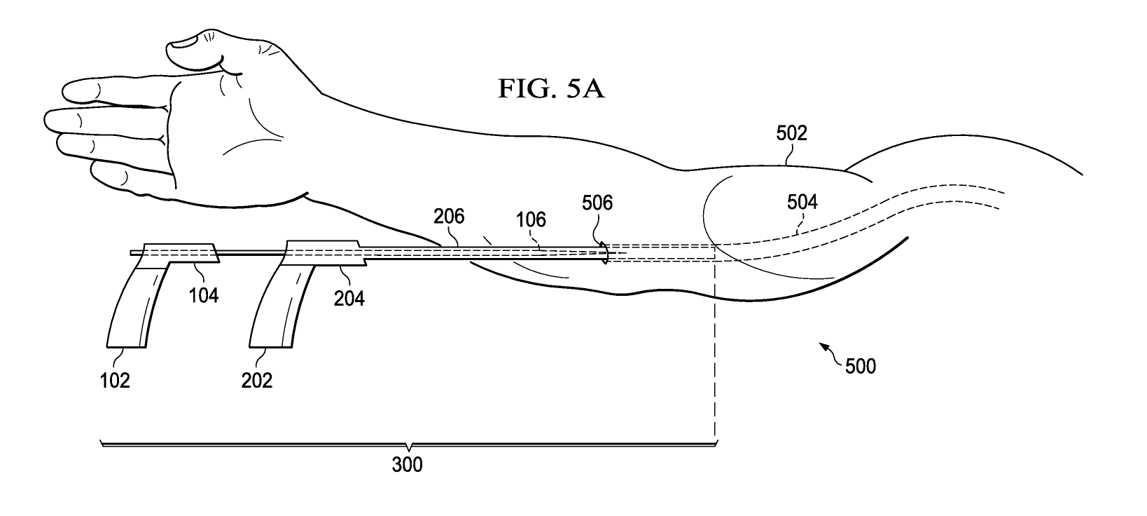

In FIG. 4, rod handle 102 has been pushed forward against sheath handle 202 so that the distal end 108 of rod body 106 exits sheath body 200. Rod handle 102 is pushed so that rod body 206 and rod housing 204 slide longitudinally through sheath 200 until rod handle 102 is prevented by sheath handle 202 and sheath housing 204 from moving any further. Once distal end 108 (which normally is curved) reaches the end and exits sheath body 206, distal end 108 immediately returns to its original curved shape because sheath body 206 will no longer be asserting a force against distal end 108 to keep distal end 108 straight. Thus, at this point, distal end 108 will be radially curved approximately 90 degrees from the longitudinal axis of the rest of rod body 106 and sheath body 206.

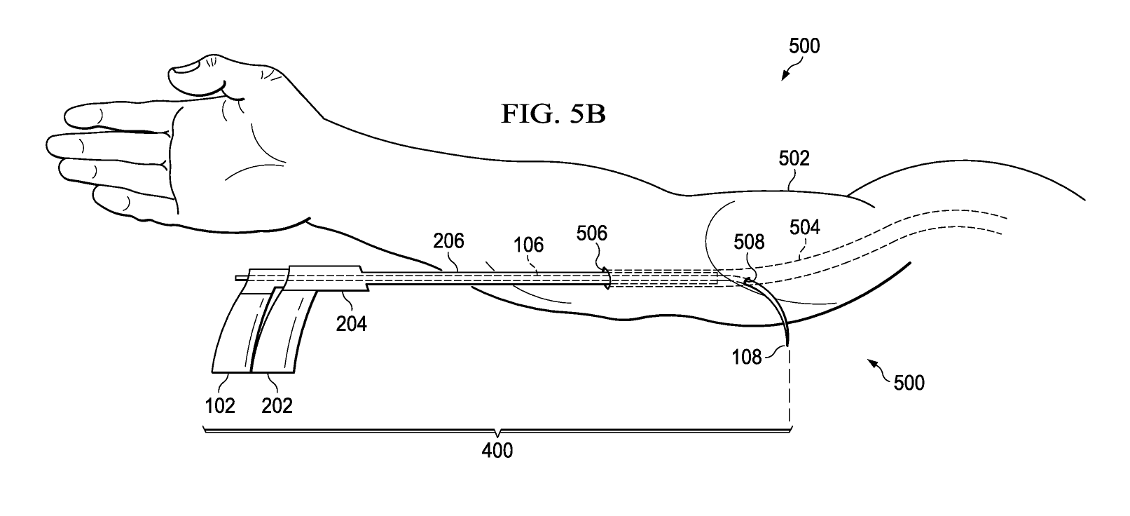

FIGS. 5a and 5b illustrate an exemplary technique in which an embodiment of the medical device is being used on patient 500, in accordance with various embodiments of the invention. While exemplary FIGS. 5a and 5b depict the combination of rod 100 and sheath 200 being used in patient 500's arm 502, the combination may be used anywhere in patient 500. FIG. 5a shows patient 500 with vascular access device 300 in which the rod body 106 is fully disposed within the sheath body 206. Rod body 106, entirely disposed within sheath body 206, is inserted through an insertion 506 into a blood vessel 504. Vascular access device 300 is then pushed, using sheath handle 202, as far into arm 500 as desired for a second insertion.

In FIG. 5b, at the point in which a second insertion is desired, rod handle 102 is pushed forward toward sheath handle 202 thereby forcing rod housing 104 and rod body 106 to move longitudinally with respect to sheath 200. By forcing the rod handle 102 and sheath handle 202 together, distal end 108 of rod body 106 exits sheath body 206 and immediately returns to its original curved shape, thereby puncturing the skin in arm 502 to make second insertion 508. Distal end 108 punctures arm 502 in the same direction as rod handle 102 extends axially from rod housing 104.

Some of these embodiments allow a physician to make a second insertion on a patient from within the patient himself/herself thereby allowing the physician to gain vascular access to the patient in a much more efficient manner than having to make the second insertion from outside the patient (which may be difficult due to location and size of the patient's extremities). Because the rod body 106 is hollow, a wire may be threaded through it, allowing the physician the capability of easily guiding and inserting a second sheath in the opposite direction in order to make the access functional.

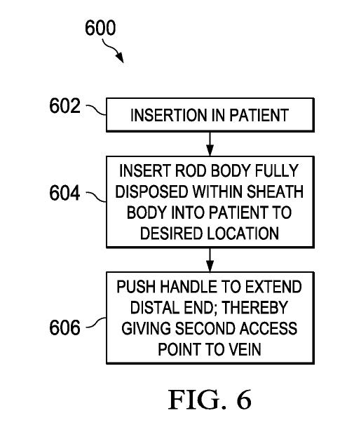

FIG. 6 shows an illustrative flow diagram of method 600 implemented in accordance with embodiments of the invention. Method 600 comprises, in block 602, making an insertion in a patient. This insertion point is preferably large enough in diameter to enable sheath body 206 from FIG. 2 to be inserted into the patient. The method continues in block 604 with insertion of rod body 106 fully disposed within sheath body 206 from FIG. 3, through the insertion point and pushed up the patient's arm until the end of sheath body 206 is at the desired location of a second insertion. Rod body 106 is fully enclosed within the sheath body 206 during this step.

Method 600 also comprises, in block 606, pushing rod handle 102, from FIG. 1, thereby forcing rod housing 104 and rod body 106, from FIG. 4, to slide longitudinally with respect to sheath body 206, from FIG. 4. At the point that rod body 106, including distal end 108, from FIG. 1, reaches the point where it is curved, distal end 108 immediately returns to its original curved shape, thereby puncturing the skin in the patient's arm to make a second insertion.

The above discussion is meant to be illustrative of the principles and various embodiments of the present invention. Numerous variations and modifications will become apparent to those skilled in the art once the above disclosure is fully appreciated. It is intended that the following claims be interpreted to embrace all such variations and modifications.

What is claimed is:

1. A medical device comprising:
a rod including an elongate rod body and a rod handle, wherein the rod body is composed of a biocompatible semi-rigid material and comprises a distal end having a point configured to puncture human skin, and wherein the rod body is curved relative to the longitudinal axis of the rod body; and
a sheath including a hollow sheath body and a sheath handle, wherein the sheath body is substantially straight, and
wherein the rod body is received into the sheath body and slides along the sheath body.

2. The medical device of claim 1, wherein the rod body is curved from between 75 to 105 degrees relative to the longitudinal axis of the rod body.

3. The medical device of claim 1, wherein the rod body is composed of a Nitinol alloy.

4. The medical device of claim 1, wherein the rod body is hollow.

5. The medical device of claim 1, wherein the sheath handle and rod handle are composed of non-slip materials.

6. The medical device of claim 1, wherein the rod body is at least 20 centimeters in length.

7. The medical device of claim 1, wherein the sheath body is at least 13 centimeters in length.

8. The medical device of claim 1, wherein the rod body is longer than the sheath body so that when the sheath handle and rod handle are pushed together, the distal end exits the sheath body and curves away from the sheath body.

9. The medical device of claim 1, wherein the distal end of the rod body is superelastic.

10. A medical device comprising:
a rod including an elongate rod body and a rod handle; and
a sheath including a hollow sheath body and a sheath handle, wherein the sheath body is substantially straight and has a longitudinal axis,
wherein when the rod is in a first position within the sheath body, a distal end of the rod body is fully disposed within the sheath body, and
wherein when the rod is in a second position within the sheath body, the distal end of the rod body extends from the sheath body and away from the longitudinal axis of the sheath body.

11. The medical device of claim 10, wherein the rod body is composed of a biocompatible semi-rigid material.

12. The medical device of claim 11, wherein the rod body is composed of a Nitinol alloy.

13. The medical device of claim 10, wherein the distal end of the rod body is beveled to a point for puncturing through human skin.

14. The medical device of claim 10, wherein the distal end of the rod body is superelastic.

15. The medical device of claim 10, wherein the rod body is at least 20 centimeters in length.

16. The medical device of claim 10, wherein the rod body is hollow.

17. The medical device of claim 10, wherein the sheath body is at least 13 centimeters in length.

18. The medical device of claim 10, wherein the sheath handle and rod handle are composed of non-slip materials.

19. The medical device of claim 10, wherein when the rod is in the first position, the rod handle is horizontally separated from the sheath handle, and
wherein when the rod is in the second position, the rod handle is in contact with the sheath handle.

20. A method comprising:
making a first insertion on a patient;
inserting a rod body entirely disposed within a sheath body into the patient through the first insertion and into a blood vessel;
sliding the rod body longitudinally along the sheath body until a distal end of the rod body emerges from the sheath body, the blood vessel, and the skin of the patient at an angle relative to the longitudinal axis of the sheath body.
